(19) United States
(12) Patent Application Publication
(10) Pub. No.: US 2022/0211523 A1
(43) Pub. Date: Jul. 7, 2022

(54) LASER DIMPLED STENT FOR PREVENTION OF RESTENOSIS

(71) Applicant: University of North Texas, Denton, TX (US)

(72) Inventors: Narendra Dahotre, Corinth, TX (US); Jewon Sohn, Frisco, TX (US); Sameehan S. Joshi, Dallas, TX (US)

(73) Assignee: University of North Texas, Denton, TX (US)

(21) Appl. No.: 17/699,476

(22) Filed: Mar. 21, 2022

Related U.S. Application Data
(63) Continuation-in-part of application No. 16/494,953, filed on Sep. 17, 2019.
(60) Provisional application No. 62/475,574, filed on Mar. 23, 2017.

(51) Int. Cl. A61F 2/82; B23K 26/352

(52) U.S. Cl. CPC A61F 2/82; A61F 2240/002; A61F 2230/0069; B23K 26/355

(57) ABSTRACT

A dimpled stent design has geometrical characteristics, namely dimple width and depth, which generate dimple site specific turbulence and thrust within the blood flow to reduce or eliminate restenosis. The dimpled stent design is produced by laser processing of the stent material to produce different sizes, which may be predicted by a Multiphysics computational model, placements, and spatial layouts of dimples in the stent.

This application is a continuation-in-part of and claims priority to U.S. patent application Ser. No. 16/494,953, filed on Sep. 17, 2019, entitled “Laser Dimpled Stent for Prevention of Restenosis,” which is a 371 application of PCT/US2017/59753, filed Nov. 2, 2017, entitled “Laser Dimpled Stent for Prevention of Restenosis,” which claims priority to U.S. Provisional Patent Application Ser. No. 62/475,574, filed on Mar. 23, 2017, entitled “Laser Dimpled Stent for Prevention of Restenosis,” the entire contents of which are hereby incorporated by reference.

### BACKGROUND

The present invention relates generally to a stent designed to prevent tissue build-up after placement. In particular, the stent is subjected to laser processing to introduce one or more dimples that increase localized turbulence inside the stent and reduce the potential for tissue build-up and restenosis.

Angioplasty is a safe and effective way to unblock coronary arteries. Initially, angioplasty was performed only with balloon catheters, but technical advances have been made and improved patient outcome has been achieved with the placement of small metallic spring-like or tube-like devices called “stents” at the site of the blockage. The implanted stent serves as a scaffold that keeps the artery open. However, the restenosis rate, the recurrence of stenosis, a narrowing of a blood vessel, leading to restricted blood flow continuously increases after corrective surgery due to deposition of excessive plateaus (platelets) on the inner wall of the stent. As a result, patients have to repeatedly undergo coronary angioplasty surgery. Accordingly, increasing interest is concentrated on developing methods that will substantially lower the rate of restenosis.

Restenosis, the recurrence of abnormal narrowing of a coronary artery from scar tissue and cholesterol build-up after corrective surgery increases exponentially as the duration of time from surgery increases and eventually plateau percentages reach nearly 75% as shown in FIG. 1.

Angioplasty, the surgical removal of material build-up in a coronary artery and subsequent implant of a coronary stent, poses two significant disadvantages: the widened surface of the artery becomes uneven due to the uneven expansion of the catheter tube, and the inner surface of the artery displays recoil by springing back to its original position to a certain degree after the catheter has been removed, increasing the risk of arterial blockage from material build-up.

The disadvantages of angioplasty lead to several issues in present clinical settings: (1) the repeated, dangerously detailed replacement of a coronary stent, (2) the extreme inefficiency of the stent, and consequently, (3) the dramatically increased cost for the patients and practitioners alike. In light of this, the research community is focused on developing an improved stent design and the present investigation forms a part of such an effort.

Efforts have been made to diminish the adverse side effects of conventional stents. Various specialized stents were formulated to target a specific disadvantage of the results from an angioplasty. The bare metal stent (BMS) (shown in FIG. 2), a tubular wired mesh stent with no coating, is used to lower the rate of thrombosis (formation of blood clot) once tissue regrowth covered the stent. However, there are several side effects for its use, including arterial perforation or rupture, coronary artery spasm, cerebrovascular accident, heightened rate of restenosis, and unstable angina pectoris.

The drug-eluting stent (DES) (also shown in FIG. 2), consists of a metallic backbone, a situation-specific anti-proliferative drug, and a vehicle polymer that controls drug release rate, reduces restenosis and revascularization. This stent releases a drug geared toward inhibition of neointima, which causes restenosis. However, utilization of the drug-eluting stent induces a lengthy duration (approximately twelve months) of dual antiplatelet therapy (a combination of Aspirin and P2Y12 receptor blocker) in order to preclude an already higher rate of thrombosis.

Some commercially available bioengineered dual therapy stents (DTS) specifically target arteries of patients with contraindications for the dual antiplatelet therapy that is a part of the drug-eluting stent. One example of such a stent is coated with an antibody that captures CD34+ endothelial progenitor cells to the stent and the subsequently formed endothelial layer protects against thrombosis and decreases restenosis. A major disability in this stent is its lack of rigidity. Thus, DTS combines the benefit of DES and bio-engineered stents and is the only stent to contain a drug with active healing technology. Compared to these bioengineered stents, others embody more structure within their frames, restricting restenosis and supporting the internal arterial walls.

Biodegradable vascular scaffold stents (BVS) (shown in FIG. 3) are a type of drug-eluting stent on a dissolvable scaffold platform. One such stent is coated with paclitaxel which is released from a polymer and both are absorbed by the body as time progresses, reducing irritation of the arterial lumen. The scaffold itself is absorbed over time. Unlike with the DTS, there is no active element to promote artery healing. The biodegradable vascular scaffold stent contains thicker struts compared to the bare metal stents. This causes the stent to bear less tolerance for the possible overexpansion of the mesh.

Finally, the dual therapy stents are considered to be the most advanced model because of their drug-release mechanism that lead to reduction in inflammation, and arterial healing. A biodegradable polymer releases a drug that significantly reduces restenosis. However, similar to the bioengineered stents, the dual therapy stents also exhibit increased rigidity within the structure thereby inhibiting their ability to accommodate the artery's intrinsic flexibility.

All of the current state-of-the-art stents demonstrate common disadvantages such as inefficiency, instability, and higher cost. Thus, improved stent design in order to control restenosis in a feasible manner is still being explored.

### SUMMARY

The present disclosure relates to an innovative dimpled stent design and its fluid flow characteristics. Its geometrical characteristics, namely dimple width and depth are likely to generate localized turbulence and thrust within the blood flow site specific to the dimple, a direct result of the Magnus Effect to keep the plateaus (platelets) flowing with the blood stream without sticking to and the depositing on the wall of stent as scar tissue and cholesterol. Such a physical phenomenon is mathematically expressed as

$$\vec{{F}_{M}} = \frac{1}{2} {C}_{L} ρ {Aυ}^{2} ( \hat{ω} \times \hat{v} ) \qquad ( 1 )$$

Where \vec{F_{M}} is the Magnus force, \hat{ω} is the vector representing an axis of rotation, \vec{ν} is the velocity vector of the flow, C_{L} is the lift coefficient, ρ is the density of the blood, A is the cross-sectional area of the platelet, v is the platelet velocity.

A dimpled stent design mirrors the working principle of a golf ball. Drag on a golf ball results mainly from air pressure forces, arising when the pressure in front of the ball is significantly higher than that behind the ball. The only practical way of reducing this difference is to design the ball surface to attract the main stream of air flowing by it as close to the surface of the ball as possible. This situation is achieved by the golf ball's dimples, which augment the turbulence close to the surface, bringing the high-speed air stream closer and increasing the pressure behind the ball. The effect is plotted in the chart, which shows that for Reynolds numbers (Re) achievable by hitting the ball with a club (10⁵), the coefficient of drag C_{D} is much lower for the dimpled ball, as shown in FIG. 4. With the decreased drag of the golf ball, the golf ball projects further and the air flow around it accelerates.

Similar to the mechanics behind a golf ball's dimples and the surrounding fluid flow, the fluid flow in proximity of the inner stent surface dimples accelerate just enough to overcome the threshold that increases the rate of restenosis due to accumulation of decelerated platelets. The dimpled stent design simultaneously grants the possibility of extending the life of the stent while maintaining a stable and healthy blood velocity.

Based on the dimpled golf ball concept, a similar design is used for a stent application. Such a design based on basic fluid flow concepts would create a high pressure (thrust) zone and an Eddy zone within the dimpled region of the stent, as shown in FIG. 5. Furthermore, such a fluid flow would cause the platelets to flush away from the stent walls providing a possibility to prolong the stent life, as shown in FIG. 6. Moreover, the concept and working principle of a single dimple stent design can be extended to a multi-dimple design.

The dimpled stent design provides a fundamental improvement within the structure of state-of-the-art stents, allowing new possibilities to be explored in terms of sustainability in the field of cardiology. The new ability to reduce or eliminate restenosis will allow physicians to focus on efficient treatment of angina rather than attempting to prevent its recurrence. The focus can shift, for example, from restenosis prevention to drug optimization. As drug optimization is just one example, the extent of new capabilities presented by engineering of the dimpled stent is essentially boundless in that any stent can be personalized to the patients' conditions. The combination of the drug release and its Ti6Al4V base metal allow for non-toxic, bio-friendly metals, ensuring the safety of the patient.

It is notable that the turbulence in fluid flow resides chiefly within the dimple, causing minimal disturbance in the main flow. Thus, the dimple generates just enough turbulence to ultimately restrain restenosis without potentially causing discomfort for the patient.

Different patterns and diverse architecture of dimples can be utilized. These include staggered, diagonal, in-lined, and spiral patterns in addition to the lay outs illustrated in FIG. 7. The various patterns listed differ in three key aspects: pitch P, the spacing between centers of two consecutive (adjacent) dimples in a given linear or spiral row of dimples, pitch R, the spacing between centers of two consecutive (adjacent) dimples in two given consecutive (adjacent) linear or spiral rows of dimples respectively, and orientation θ, the angle between pitch P and pitch R. Specifically, the staggered and spiral arrangements will display longer pitches compared to the in-lined and diagonal arrangements. The subsequent differences in pitches affect the fluid flow around each individual dimple through turbulence and friction factors.

The laser ablation of coronary stents to produce dimples on the inner surface essentially is a cost-effective, stable, and adept method to eliminate arterial narrowing recurrences after corrective surgery. The dimpled stent design deals with the issues such as inefficiency, instability, and the cost of repeated coronary angioplasty surgery currently unsolved by state-of-the-art stents.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows restenosis rates in coronary arteries at 8 months and 4 years following corrective surgeries (courtesy of Jorgensen et al. 2009).

FIG. 2 shows a diagram of thrombus formation in an artery having a drug-eluting stent (DES) and restenosis in an artery having a bare-metal stent (BMS) (courtesy of Shah 2003).

FIG. 3 shows a schematic of a biodegradable vascular scaffold (BVS) and the events that occur after insertion of the scaffold (courtesy of Balachandran et al. 2015).

FIG. 4 shows variation in drag coefficient (C_{D}) of a smooth surface ball, rough surface balls and a golf ball as a function of Reynolds number (Re) where s is the sand grain roughness height and m is the ball diameter (courtesy of Bearman et al. 1976).

FIG. 5 shows a schematic of a blood flow pattern in a dimpled stent having a dimple width w and depth d with high pressure and eddy zones within the dimple.

FIG. 6 shows a schematic of flushing of platelets in a dimpled stent having dimple diameter d where the platelets do not stick to the wall due to the turbulent flow and thrust generated within the dimple.

FIG. 7 shows schematics of (a) and (b), two exemplary placements (or layouts) of dimples in a stent design in accordance with preferred embodiments described herein, and (c) exemplary employment of dimples of various diameters in a stent design in accordance with preferred embodiments described herein.

FIG. 8 shows a computational simulation of a dimple produced on Ti6Al4V surface with a laser of 500 W and 0.12 s exposure time including (A) evolution of the ablation depth and (B) corresponding time temperature profiles.

FIG. 9 shows a computational simulation of a dimple produced on Ti6Al4V surface with a laser of 700 W and 0.12 s exposure time, including (A) evolution of the ablation depth and (B) corresponding time temperature profiles.

FIG. 10 shows a computational simulation of a dimple produced on Ti6Al4V surface with a laser of 900 W and 0.12 s exposure time, including (A) evolution of the ablation depth and (B) corresponding time temperature profiles.

FIG. 11 shows a computational simulation of a dimple produced on Ti6Al4V surface with a laser of 1800 W and 0.12 s exposure time, including (A) evolution of the ablation depth and (B) corresponding time temperature profiles.

FIG. 12 shows vaporization depths within the dimple of a stent material as a function of laser input power for constant beam residence time of 0.12 s.

FIG. 13A shows a side view of a dimpled stent design in accordance with a preferred embodiment.

FIG. 13B shows an end view of a dimpled stent design in accordance with a preferred embodiment.

FIG. 14 shows a schematic of an experimental setup used for evaluating gravity fed SBF flow characteristics of exemplary dimpled stent material.

FIG. 15 shows different times of fluid (SBF) flow characteristics under various test conditions.

FIG. 16 shows average fluid flow velocity for various test conditions.

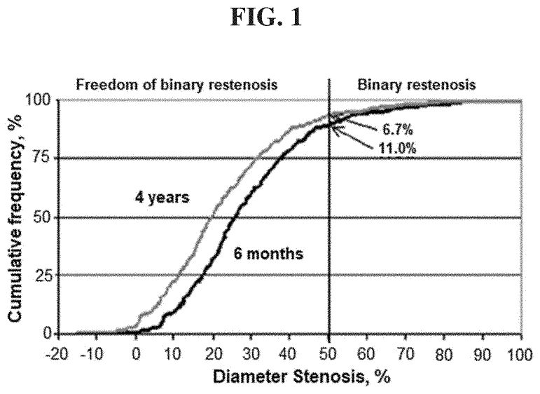

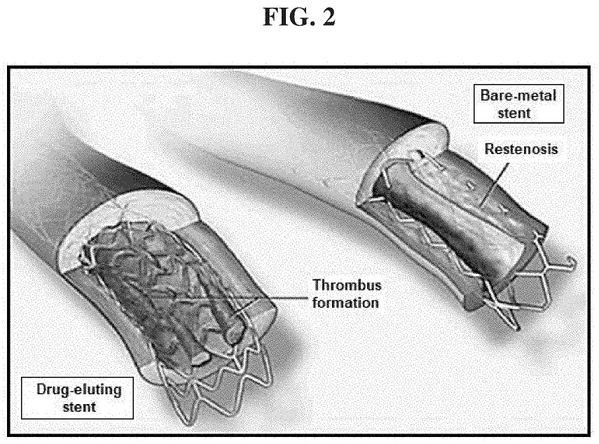

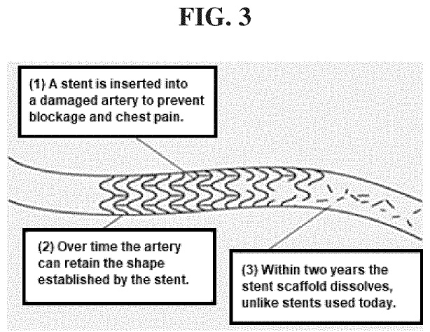

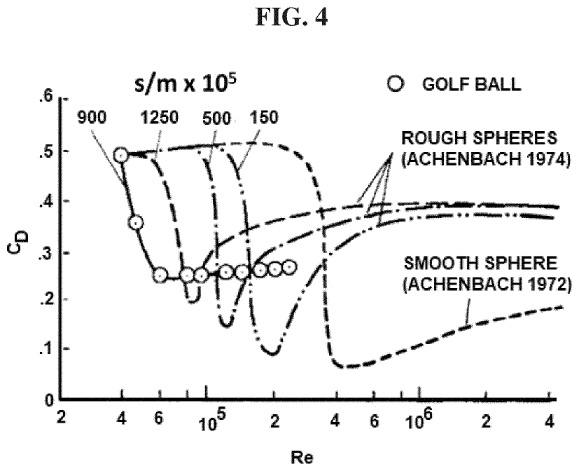

### DETAILED DESCRIPTION

The present disclosure pertains to a stent having a dimpled texture which helps to eliminate arterial narrowing recurrences after corrective surgery.

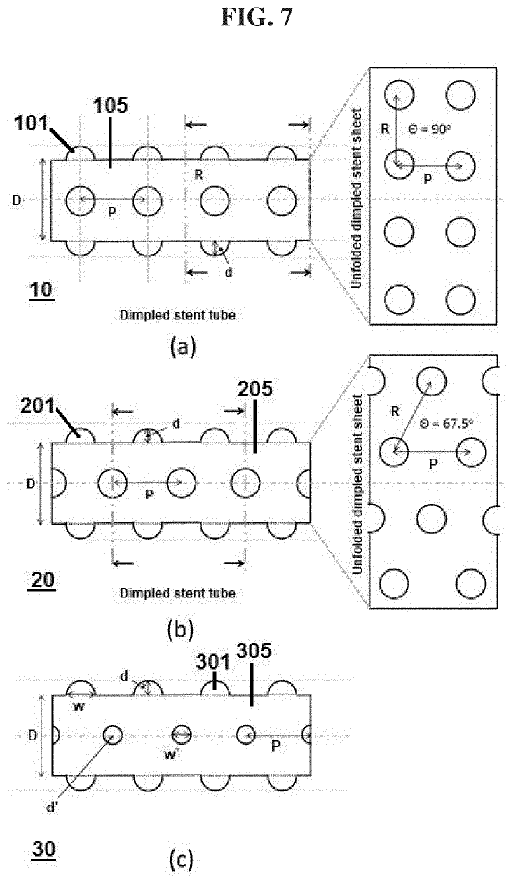

FIG. 7 shows schematics of three types of placements (lay outs) of dimples in preferred embodiments of a stent design. D is the diameter of the stent, d is the depth of the dimple, w is the width of the dimple, and the pitch P is the spacing between centers of two consecutive (adjacent) dimples in a given linear or spiral row of dimples. P preferably ranges between 1.8 w and 2.2 w to maintain the required turbulence and the thrust. The pitch R is the spacing between centers of two consecutive (adjacent) dimples in two given consecutive (adjacent) linear or spiral rows of dimples, where R preferably ranges between 1.8 w and 2.2 w. In case of the placement with dimples of two different widths of w and w′ in each consecutive (adjacent) linear row or spiral row, P ranges between 1.8 times (w+w′) and 2.2 times (w+w′). FIG. 7(a) shows a preferred embodiment of a dimpled stent 10 having dimples 101 in a stent material 105 with a dimple placement where θ=90° where θ is the angle between P and R vectors and ranges between 0° and 90°. FIG. 7(b) shows a preferred embodiment of a dimpled stent 20 having dimples 201 in a stent material 205 with a dimple placement where θ=67.5°. Combinations of orientation θ between P and R, along with w, w′, P, and R would yield various embodiments of stent designs. The parameters such as center to center pitch of the dimple placement (P), center to center spacing between horizontal stent rows (R) and orientation (θ) between P and R can be varied to generate various combinations of dimple placements (layout) on the stent surface. FIGS. 7(a) and 7(b) show the embodiments of dimpled stents 10 and 20 in both a folded tube shape and in an unfolded dimpled stent sheet, which would be formed into a tube sheet to create the dimpled stent. FIG. 7(c) provides one such specific layout for a dimpled stent 30 with different widths (w and w′) and depths (d and d′) in alternate rows of dimples 301 in a stent material 305 and the orientation, θ, between pitches P and R equal to 67.5°.

The magnitude of dimple site specific turbulence and thrust within the blood flow depends on several parameters such as but not limited to dimple morphology: spherical (width and depth) or elliptical (major and minor axes and depth); geometric layout of the dimple pattern; and number of dimples. These parameters can be precisely fabricated and controlled via the laser ablation of Ti6Al4V (a commonly used biocompatible titanium alloy), while employing a variety of laser processing parameters, including laser power, traverse speed, and laser beam diameter on the surface of the material which together provide desired input laser fluence.

The disparate architectural dimensions of the dimples that were modeled through the Finite Element (FE) based Multiphysics computational platform display correlation between the presence of dimples of required morphology (width and depth in case of the spherical dimple) for increased fluid (blood) flow and thrust that in turn drastically decrease the adhesion of platelets and formation of plaque in the interior wall. During in vivo study, the above dimensions of the stent dimples accelerated the overall flow of simulated body fluid (SBF) (a solution consisting of the same chemical composition as that of blood without the plasma), thereby providing a way to potentially substantially diminish the rate of restenosis. The innovative approach adopted is expected to increase the efficiency and lifespan of the stent enhancing the life of patients.

A computational model was developed in house and employed to model the interaction between the heat source (laser) and the titanium alloy (Ti6Al4V) surface during creation of a dimple of desired spherical geometry (width and depth). During laser processing (dimpling), material experiences various physical phenomena such as phase transition from solid-to-liquid-to-vaporization and material loss during evaporation. In addition, the material surrounding dimpled region also experiences the transition dependent effects such as thermal expansion during heating, recoil pressure during vaporization, and Marangoni convection and surface tension during phase transition. In order to simulate such complex laser dimpling mechanism, the present model was designed and developed using multistep and Multiphysics computational modeling approach for multidimensional (3D) laser dimpling process on a finite-element (FE) platform. The computational model based on the Multiphysics approach combines heat transfer, fluid flow, and structural mechanics for thermo-mechanical coupling (temperature and thermal expansion coefficient) along with several forces such as body force (gravitational force) and surface forces (viscosity/shear forces, recoil pressure) to investigate the combinatorial effects of these physical phenomena on evolution of physical attributes/surface topography (depth, width, and geometry) of a dimple. The selective governing equation based on heat transfer for a preferred Multiphysics computational model is presented in Eq. (2).

$$ρ {{C}_{p} [ \frac{\partial T}{\partial L} ]}_{( x , y , z )} = k \{ {[ \frac{{\partial }^{2} T}{\partial {x}^{2}} ]}_{( y , z , t )} + {[ \frac{{\partial }^{2} T}{\partial {y}^{2}} ]}_{( z , x , t )} + {[ \frac{{\partial }^{2} T}{\partial {z}^{2}} ]}_{( x , y , t )} \} \qquad ( 2 )$$

Here, k is the thermal conductivity, Cₚ is the specific heat and ρ is the density of the material. Furthermore, the laser material interaction region is assigned a heat flux boundary with a moving laser beam defined by Eq. (3).

$$k [ ( \frac{\partial T}{\partial x} ) + ( \frac{\partial T}{\partial y} ) + ( \frac{\partial T}{\partial z} ) ] = {P}_{X} - \{ h [ T - {T}_{0} ] + ɛ σ [ {T}^{4} - {T}_{0}^{4} ] \} \qquad ( 3 )$$

Here, h is heat transfer coefficient, ε is emissivity, σ is Stefan-Boltzman constant, T₀ is ambient temperature, and P_{X} is the input laser power intensity distribution. Such a heat source, P, can have Gaussian or top hat or a dumbbell shaped beam profile in terms of spatial distribution of the power as expressed in the following set of equations.

$$\begin{matrix}{P}_{g} = \frac{{P}_{0}}{π {r}_{0}^{2}} {exp}^{- ⌊ \frac{{x}^{2} + {y}^{2}}{{r}_{0}^{2}} ⌋} & ( 4 ) \\ {P}_{th} = \frac{{P}_{0}}{π {r}_{0}^{2}} {exp}^{- \frac{{( {x}^{2} + {y}^{2} )}^{n}}{{r}_{0}^{2}}} ( where n -> ∞ ) & ( 5 ) \\ {P}_{db} = \frac{2 {P}_{0}}{π {r}_{0}^{2}} {( \frac{x + y}{{r}_{0}} )}^{2} {exp}^{- \frac{{( {x}^{2} + {y}^{2} )}^{n}}{{r}_{0}^{2}}} & ( 6 )\end{matrix}$$

Where P_{g} is the Gaussian heat flux, Pₜₕ is the top hat heat flux, P_{db} is the dumbbell heat flux, P₀ is laser input power, r₀ is the radius of beam at which laser power transverse intensity decreases to 1/e², and x, y, z are the Cartesian coordinates with y along the axis of the beam and x and z are in the plane orthogonal to the axis of the beam and the beam intensity distribution is considered axisymmetric in x-z plane.

All other surfaces are assigned convective cooling and surface to ambient radiation boundary conditions given by the following relationship (Eq. (7)):

$$- k [ ( \frac{\partial T}{\partial x} ) + ( \frac{\partial T}{\partial y} ) + ( \frac{\partial T}{\partial z} ) ] = h [ T - {T}_{0} ] + ɛ σ [ {T}^{4} - {T}_{0}^{4} ] \qquad ( 7 )$$

Moreover, there are various types of lasers available commercially which could be employed for stent surface ablation to produce a dimpled texture on it. These lasers have various wavelengths within the electromagnetic spectrum. Moreover, depending upon the laser type under consideration, a pulsed and/or continuous wave modes of operation can be adopted. In case of pulsed lasers, the pulse duration during interaction with the material can range from femtosecond (10⁻¹⁵ s) to millisecond (10⁻³ s). Such characteristics of commercial lasers that can be adopted in laser dimpling of the stent are tabulated below in Table 1. The basic classification of such lasers in infrared and ultraviolet type is based on the wavelength range in which they operate.

TABLE 1
Various types of lasers and their characteristics
suitable for producing dimpled stent material

| | | |
|---|---|---|
| Type of laser | Wavelength (nm) | Interaction time |

Infrared Lasers

| | | |
|---|---|---|
| CO₂ | 9600-10600 | micro seconds to |
| | | continuous wave |
| YAG | 232, 532, 1064 | fempto seconds to |
| (eg: Nd: YAG, Er: | | continuous wave |
| YAG, and NdCrYAG) | | |
| Ti: Sapphire | 650-1100 | fempto seconds to |
| | | continuous wave |
| Diode laser | 900-1100 | mili seconds to |
| | | continuous wave |
| Free electron laser | tunable wavelength | Micro to mili |
| | | seconds |

Ultraviolet lasers

| | | |
|---|---|---|
| Excimer lasers | Ultraviolet range including | fempto to nano |
| (eg: ArF, KrF, | 157, 248, 282, 308, 351 | seconds |
| XeBr, and XeCl) | depending upon the gaseous | |
| | laser medium employed | |

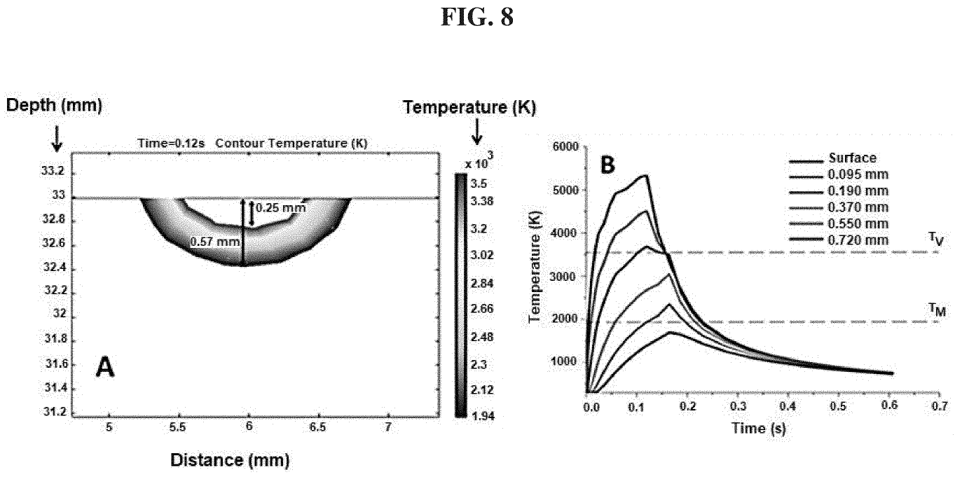

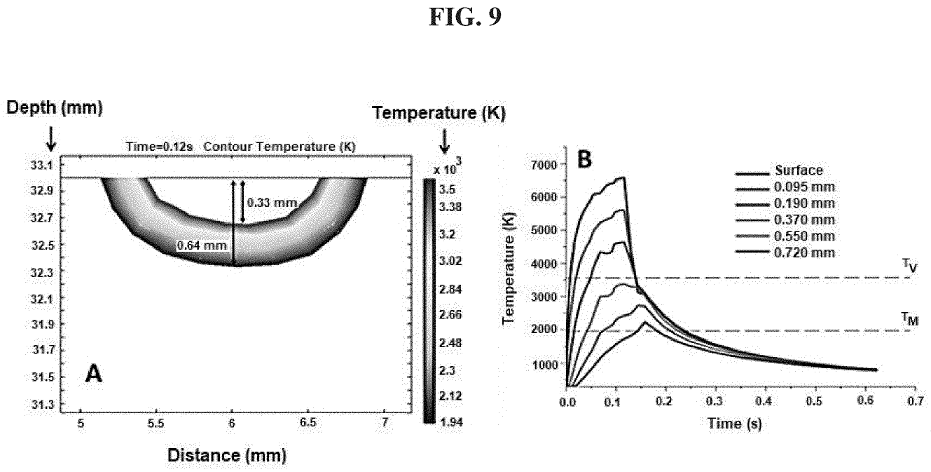

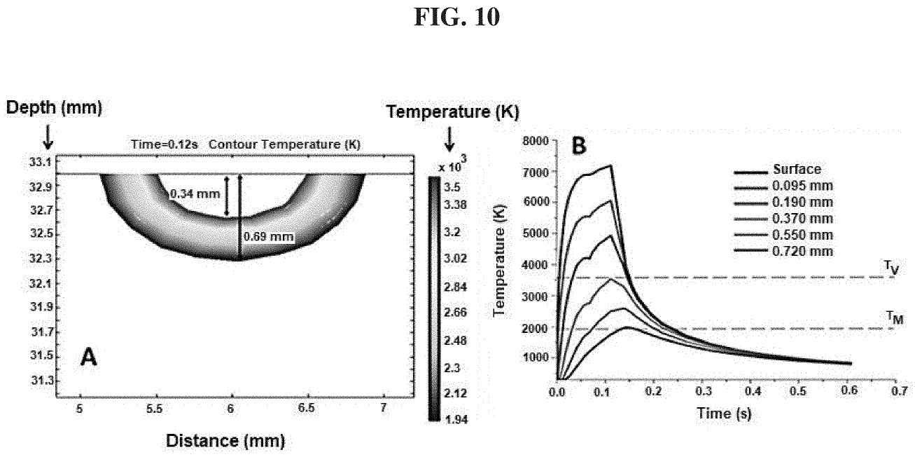

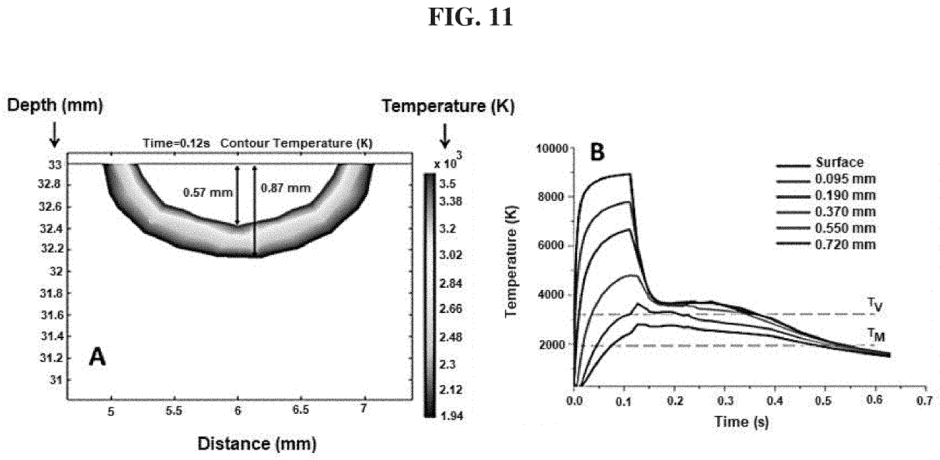

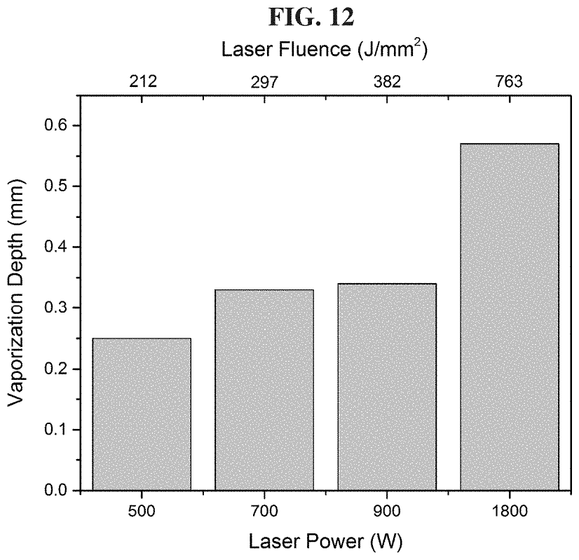

During the present preliminary efforts, feasibility of the suitable laser processing parameters for laser-based dimpling of the stent material (Ti6Al4V) was established through the computational model. The evolution of the dimple morphology was established through a time-dependent model parameters for the discrete laser residence time (the length of time of laser-material interaction) of 0.12 seconds by incorporating material properties such as density, specific heat, and thermal conductivity along with consideration to the physical phenomena such as the temperature dependent transition of material from solid to liquid to gas. Thus, with the computational model based on FE simulation, the depth and width of the dimples were estimated for laser powers of 500 W, 700 W, 900 W, and 1800 W and laser residence time of 0.12 seconds. These computational estimations also included the optical energy density (laser fluence) delivered per unit area under these conditions. The laser fluences for the input powers used in the current work with increasing order of the power were 212, 297, 382, and 763 J/mm² respectively. The transient effects during laser dimpling are realized through computational estimations of temperature at the surface and below the surface at depths of 0.095 mm, 0.190 mm, 0.370 mm, 0.550 mm, and 0.720 mm. Some of these depths represent the exact boundaries of phase changes (solid to liquid and liquid to vapor). The boundary between the liquid phase and vapor phase represents the profile of the final dimple depth. The dimple being part of a circular geometry, the width and depth of this boundary are the dimensions of the resultant dimple. These computational simulations indicated that the formation of dimples of various morphologies (depth and width) under the laser input powers employed in the current efforts in the range of 500 W-1800 W (with corresponding laser fluence range of 212 J/mm²-763 J/mm²) occur during laser beam residence time of 0.12 seconds (shown in FIGS. 8, 9, 10, and 11). The depth and width ranges of the dimples formed is between 0.25 mm-0.57 mm and 0.9 mm-3.2 mm respectively (shown in FIG. 8-12). Thus, the computational model assisted in designing a combination of the process parameters (laser power, laser-material interaction time) and desired/resultant dimple parameters (depth and width). Furthermore, it has been reported in the literature that the optimum ratio of dimple depth (d or d′) to dimple width (w or w′) is in the range of 0.15-0.30. In the current set of experimental conditions, laser input power of 1800 W, laser beam residence time of 0.12 seconds (corresponding laser fluence of 763 J/mm²) possessed a dimple depth to width ratio of ˜0.0.18 pointing towards potentially optimum performance. In addition, the optimum relative pitch, Q, defined as ratio of dimple spacing or pitch, P, and dimple width is reported to be in the range of 0.81-1.21. For the laser operating power of 1800 W and laser beam residence time of 0.12 seconds (laser fluence: 763 J/mm²), the estimated dimple spacing or pitch, P, has value of 3.8 mm for the Q value of 1.21. Moreover, dimples of the various widths can be employed provided the ratio of dimple width (w or w′) to dimple depth (d or d′) and pitch, Q, of the dimple are maintained in the range of 0.15-0.3 and 0.81-1.21 respectively, as shown in FIG. 7 (c).

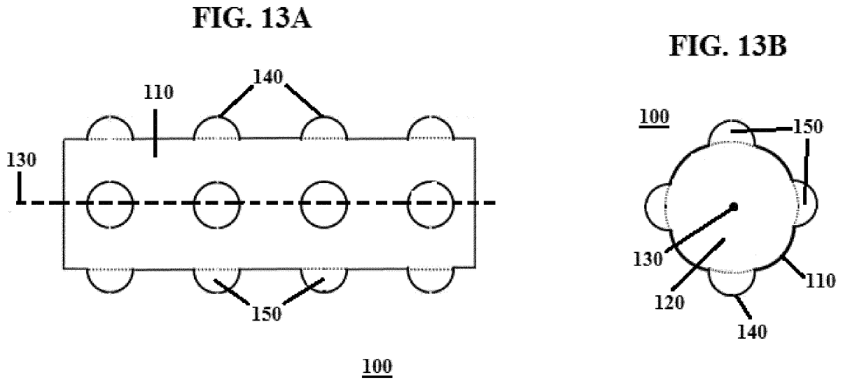

FIGS. 13A and 13B show preferred embodiments of the present invention relating to a stent 100 having circumferential walls 110 enclosing a generally cylindrical inner space 120 surrounding a central axis 130. FIG. 13A shows a view from the side of stent 100, while FIG. 13B shows a view of an end of stent 100, looking along central axis 130. The circumferential walls 110 have one or more laser produced dimples 140 protruding outwardly away from the central axis 130 to create dimpled spaces 150 in outer edges of the cylindrical inner space 120. When body fluid such as blood passes through the cylindrical inner space 120 it encounters the dimpled spaces 150 in the laser produced dimples 130 and becomes turbulent in those spaces, resulting in a decreased tendency for build-up of circulating material such as platelets against the inside of the circumferential walls 110 of the stent.

In preferred embodiments, the dimpled stent is made of any suitable material having good corrosion resistance and biocompatibility, such as a titanium alloy, cobalt-chrome alloys (MP35N), cobalt-nickel-chromium-molybdenum (CoNiCrMo) alloy, cobalt-chromium-tungsten-nickel (CoCrWNi) alloy known as L-605, stainless steel, Nitinol and biopolymers such as poly-lactic acid (PLLA), tyrosine polycarbonate, poly (anhydride ester) salicyclic acid and polytyrosine. In additional preferred embodiments, the dimpled stent is about 30 mm long, about 3.5 mm in diameter, and the circumferential walls are about 0.2 mm thick. The dimples can preferably have a depth of about 0.12 mm and a width of about 0.6 mm.

Additional preferred embodiments include a method for creating a dimpled stent. A flat coupon of stent material is preferably dimpled at one or more selected locations using a laser having a selected power and pulse interaction time to generate one or more dimples having a particular depth and width, with various spatial lay outs and various combinations of values for pitches P and R and orientation θ. The laser may be a Neodymium-Doped Yttrium Aluminum Garnet (Nd:Y₃Al₅O₁₂) or Nd:YAG laser having a 1.064 μm wavelength and fiber optic beam delivery with pulse interaction time of about 0.12 seconds and power ranging from about 500 W to 1800 W. Any other lasers listed in Table 1 can also be employed for this purpose and in that case power and interaction time can be varied in the rage of 100 W to 2000 W and 10 ns to 0.12 s, respectively. During laser processing, at a high level, the stent material contacted by the laser is partially ablated and the surrounding material changes shape into a generally dome-shaped dimple (having either spherical or ellipsoidal dome shape). After dimpling, the flat coupon of stent material, now dimpled, is mechanically formed into a cylindrical shape with the dimples protruding outwardly and two free edges of the cylindrically formed tube are precision laser butt welded (joined), for placement in a patient. Preferred applications involve use in coronary arteries following corrective surgery. Additional applications include implantation in native coronary arteries of the appropriate size, as well as implantation in a particular lesion such as proximal, non-angulated lesions, lesions of tortuous anatomy and complex situations including ostial lesions, bifurcation lesions, and calcified lesions.

In preferred embodiments, during the dimpling process, the laser material interaction region is assigned a heat flux boundary with a moving laser beam defined by:

$$k [ ( \frac{\partial T}{\partial x} ) + ( \frac{\partial T}{\partial y} ) + ( \frac{\partial T}{\partial z} ) ] = {P}_{X} - \{ h [ T - {T}_{0} ] + ɛ σ [ {T}^{4} - {T}_{0}^{4} ] \}$$

where h is heat transfer coefficient, ε is emissivity, σ is Stefan-Boltzman constant, T₀ is ambient temperature, and P_{X} is the input laser power intensity distribution. Such a heat source, Pₓ can have Gaussian or top hat or a dumbbell shaped beam profile in terms of spatial distribution of the power as expressed in the following set of equations.

$$\begin{matrix}{P}_{g} = \frac{{P}_{0}}{π {r}_{0}^{2}} {exp}^{- ⌊ \frac{{x}^{2} + {y}^{2}}{{r}_{0}^{2}} ⌋} \\ {P}_{th} = \frac{{P}_{0}}{π {r}_{0}^{2}} {exp}^{- \frac{{( {x}^{2} + {y}^{2} )}^{n}}{{r}_{0}^{2}}} ( where n -> ∞ ) \\ {P}_{db} = \frac{2 {P}_{0}}{π {r}_{0}^{2}} {( \frac{x + y}{{r}_{0}} )}^{2} {exp}^{- \frac{{( {x}^{2} + {y}^{2} )}^{n}}{{r}_{0}^{2}}}\end{matrix}$$

where P_{g} is the Gaussian heat flux, Pₜₕ is the top hat heat flux, P_{db} is the dumbbell heat flux, P₀ is laser input power, r₀ is the radius of beam at which laser power transverse intensity decreases to 1/e², and x, y, z are the Cartesisan coordinates with y along the axis of the beam and x and z are in the plane orthogonal to the axis of the beam and the beam intensity distribution is considered axisymmetric in x-z plane.

### EXAMPLES

The following examples are included to demonstrate preferred embodiments of the invention. It should be appreciated by those of skill in the art that the techniques disclosed in the examples that follow present techniques discovered by the inventor to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Example 1

Laser Fabrication of Dimpled Surface

For laser-engineering the dimples on stent material, the flat coupons Ti6Al4V alloy of dimensions 2.5×1.0×0.375 cm³ were cut on the slow speed diamond saw. The coupons were ground on 600 grit SiC emery paper for flatness and uniform average surface roughness of 4 μm. The coupons were cleaned with deionized water followed by alcohol prior to laser dimpling experiments. The dimples were created on these coupons using Neodymium-Doped Yttrium Aluminum Garnet; Nd:Y₃Al₅O₁₂ laser (1.064 μm wavelength and fiber optic beam delivery) with pulse interaction time (0.12 seconds) and powers (500, 700, 900, and 1800 W) as stated above. Only single dimples were produced under laser power-laser beam interaction (laser fluence) combination to just verify the effects of presence of a dimple and its geometry (width and depth) on the fluid flow characteristics. This approach was adopted to reveal/realize and verify the increased turbulence and thrust based flow due to the presence of a dimple on the surface compared to a flat surface (absence of dimple) of the stent material.

In Vivo Evaluation in Simulated Body Fluid

The effectiveness of creation of dimples on the surface of Ti6Al4V was in vivo evaluated with the flow of simulated body fluid (SBF). The detailed method of SBF preparation is state of the art and discussed in the publication by Tadashi Kokubo and Hiroaki Takadama., “How useful is SBF in predicting in vivo bone bioactivity?”, Biomaterials, 27, (2006): 2907-2915, incorporated by reference herein. The chemical composition of the SBF solution is presented in Table 2 below. A Tris (Tris-hydroxymethyl aminomethane) chemical was added as a stabilizer until the entire solution reached a stable pH of 7.45.

TABLE 2
Reagents for SBF preparation from Kokubo and Takadama

| | | |
|---|---|---|
| Reagent | Amount | Formula Weight |

| | | | |
|---|---|---|---|
| NaCl | 8.035 | g | 58.4430 |
| NaHCO₃ | 0.355 | g | 84.0068 |
| KCl | 0.225 | g | 74.5515 |
| K₂HPO₄•3H₂O | 0.231 | g | 228.2220 |
| MgCl₂•6H₂O | 0.311 | g | 203.3034 |
| CaCl₂ | 0.292 | g | 110.9848 |
| Na₂SO₄ | 0.072 | g | 142.0428 |
| 1.0_{M}-HCl | 5 | ml | — |

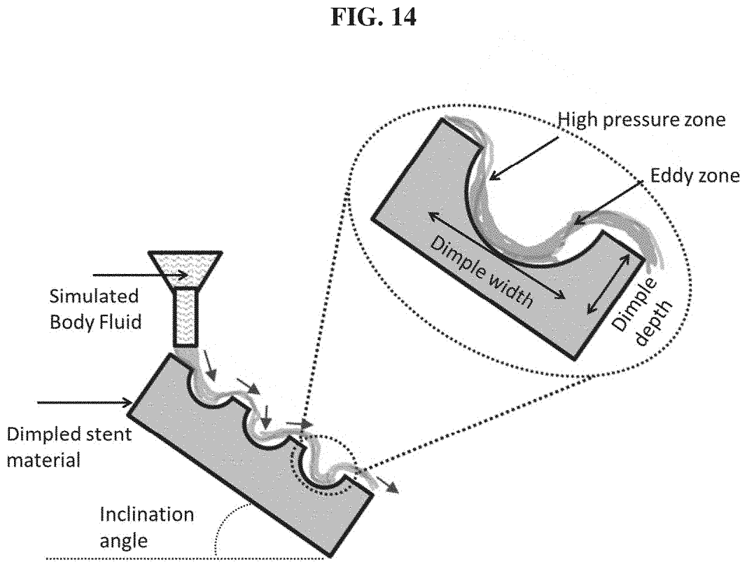

The measure of SBF flow characteristics was conducted following the general principle explained in the work of Soltani Bozchalooi, A. Careaga Houck, J. M. AlGhamdi, and K. Youcef-Toumi, “Design and control of multi-actuated atomic force microscope for large-range and high-speed imaging”, Ultramicroscopy, 160 (2016): 213-224, incorporated by reference herein. Generally, an experimental set up was designed for in vivo evaluation of SBF flow under the gravitational force over a dimpled sample, shown in FIG. 14. This assembly was put into a rubber hose to mimic the artery with the real life ratio between the length of the left subclavian artery (22-27.5 cm) and the stent (9-21 mm), averaging approximately 27:1 in size. Hence, the plastic mimic vessel used was 70 cm long, compared to the stent length of 2.5 cm. The fluid flow characteristics of gravitationally fed SBF were evaluated for time required for a given volume of SBF to flow over the sample surface of the given dimensions. For comparison, the time of flow characteristics were evaluated for three different samples of identical dimensions: (1) untreated stent material, (2) surface dimpled stent material, and (3) no stent material (empty vessel). The time for a given amount of SBF to flow through plastic mimic vessel for three different conditions of samples listed above was accurately measured using a digital watch integrated with the experimental set up. The watch accurately measured the time between the times of starting and end of fluid flow of the given volume through the mimic vessel. Total of five readings of time were recorded for each sample condition. The time of flow in combination with the distance travelled within the mimic vessel was further utilized to calculate experimentally observed SBF flow velocity.

The laser ablated volume of a dimple (dimensions) was expected to increase with increase in power from 500 W to 1800 W. In light of this, the largest dimensions (width and depth) of the dimple made with 1800 W were expected to experience the most significant changes in the fluid flow. Hence, gravitationally fed fluid flow measurements were conducted only on the samples with the dimple produced at 1800 W.

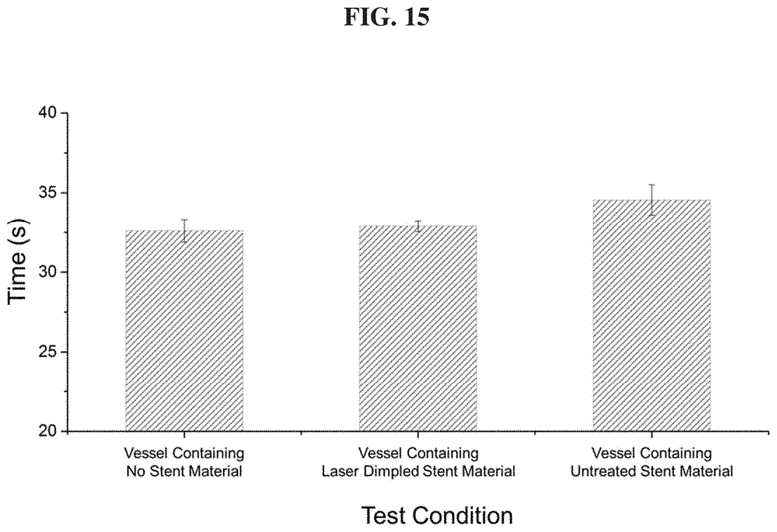

The results from the three conditions displayed clear differences in the SBF flow characteristics. As expected, the vessel with no stent displayed the shortest average time of 32.59 seconds for the SBF to flow through, shown in FIG. 15. On the other hand, the vessel with untreated stent had the average time of 34.55 seconds whereas the vessel with laser dimpled stent resulted in an average time of 32.88 seconds, also shown in FIG. 15. Thus, there was a difference of ˜2 seconds between the SBF flow in the vessel containing untreated and no stent materials. Such a difference in time was further significantly reduced to 0.3 seconds for the vessel containing laser dimpled stent. These results were encouraging with respect to the original concept and provided a possibility of deployment of the dimpled stent.

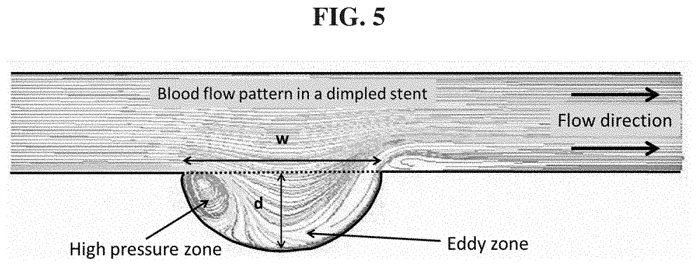

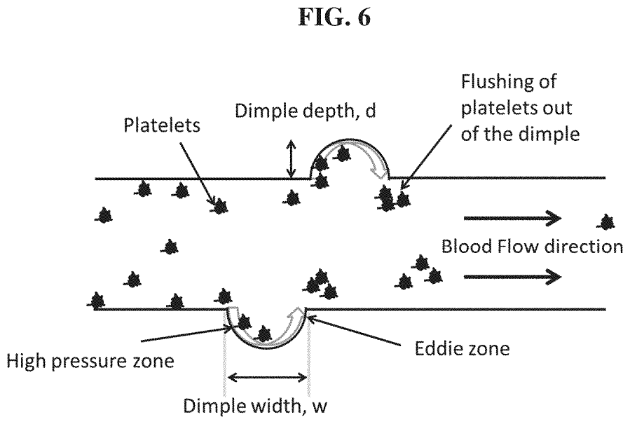

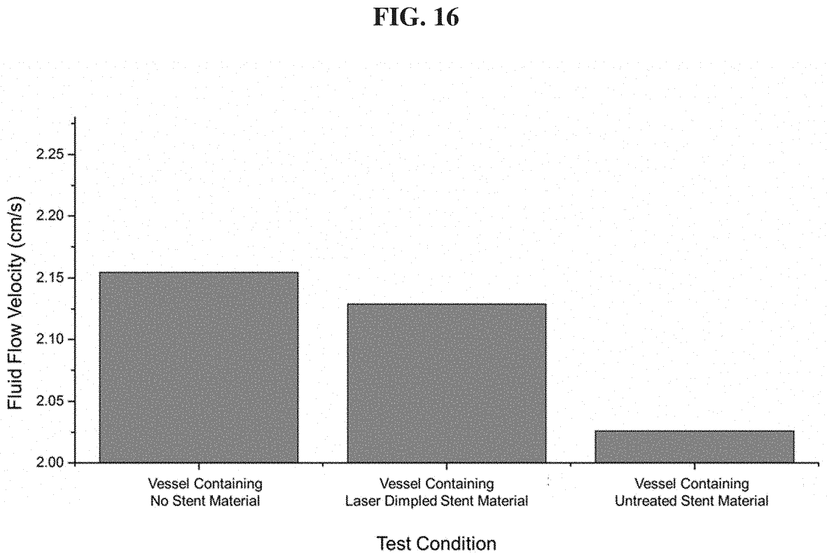

The dimensions of the dimple produced with 1800 W were predicted using computational approach described earlier. These computed dimensions were 1.62 mm and 0.60 mm for width and depth respectively. The improved fluid flow characteristic of the laser dimpled sample at 1800 W compared to non-laser treated stent material is considered to be due to increased fluid flow and thrust and decreased friction within the dimple. Such fluid flow characteristics are result of the improved theoretical fluid flow efficiency and can be realized through the formation of the vortex at the underside of the dimple and the uneven distribution of pressure inside of the dimple, both of which generate turbulence in the fluid (as shown in FIG. 5). Some portion of fluid in the dimpled zone is not easily driven by the upper main flow, and instead form an “eddy zone” that prohibits the escape of fluid (also as shown in FIG. 5). The friction factor is expected to decrease as the fluid approaches the center of the dimple and finally reaches a minimum value at the bottom. The remaining fluid in the dimpled zone that can escape is mixed with the main flow adequately. As a result of the mixing of the escaped fluid, there is a high-pressure region (see FIG. 5) at the backside of the dimpled zone causing the friction factor to reach a maximum value. The maximum friction factor value is sufficient enough to counter the sticking forces of the platelets and make them flow with the bloodstream without sticking to the stent wall as shown in FIG. 6. All of these factors are expected to result in the slight increase of fluid flow velocity in the dimpled surface in comparison to the untreated surface, shown in FIG. 16.

The preliminary efforts indicate: (1) improvement in SBF flow characteristics of the dimpled stent design compared to an untreated stent, (2) by increasing the fluid flow and thrust, the possibility of plaque formation on the stent wall can be substantially reduced, and (3) by imprinting ideal dimple dimensions and dimple spatial layout, the anti-sticking properties within the stent, the acceleration of platelets, and the subsequent decrease in the tendency for restenosis of the stent can be achieved. Although each dimple contributes to improved flow characteristics (velocity), the optimal stent dimensions and spatial layout of the dimples are critical parameters for the smoothest and most rapid blood flow (shown in FIG. 16). Furthermore, the dimpled stent can provide immensely cost-efficient disease-preventing capabilities. In conjunction with efficiency, stability, and potential minimal costs, the study indicates that laser ablation is the ideal method to fabricate a dimpled stent surface to prevent plaque formation. The reduction or the elimination of arterial blockage is expected to impact those affected by angina (chest pain) by increasing the lifetime of the stent and the patient. This design and approach are further expected to decrease the costs involved in bypass revision surgeries and improve the quality of life for the patient.

### REFERENCES CITED

The following references, to the extent that they provide exemplary procedural or other details supplementary to those set forth herein, are specifically incorporated herein by reference.
Jørgensen, Erik, Steffen Helqvist, Lene Klovgaard, Jens Kastrup, Peter Clemmensen, Lene Holmvang, Thomas Engstrom, Kari Saunamaki, and Henning Kelbæk. “Restenosis in coronary bare metal stents. Importance of time to follow-up: A comparison of coronary angiograms 6 months and 4 years after implantation.” Scandinavian Cardiovascular Journal, 43, (2009): 87-93.
Sardi. (2006). Coronary Stents. www.pages.drexel.edu/˜sr335/Background.html.
Shah, S. N. (2014). Coronary Bare-Metal Stent. emedicine.medscape.com/article/2009987-overview#a8
Shah, P. K “Inflammation, neointimal hyperplasia, and restenosis: as the leukocytes roll, the arteries thicken.” Circulation, 107 (2003): 2175-2177.
Balachandran, K., & Zambahari, R. (2015). Absorb Bioresorbable Vascular Scaffold. Institut Jantung Negara National Heart Institute.
Cottone, Robert J., G. Lawrence Thatcher, Sherry P. Parker, Laurence Hanks, David A. Kujawa, Stephen M. Rowland, Marco Costa, Robert S. Schwartz, and Yoshinobu Onuma. “OrbusNeich fully absorbable coronary stent platform incorporating dual partitioned coatings.” Euro Intervention 5, no. Suppl F (2009): F65-71
Colombo, Antonio, Goran Stankovic, and Jeffrey W. Moses. “Selection of coronary stents.” Journal of the American College of Cardiology 40, no. 6 (2002): 1021-1033.
P. W. Bearman and J. K. Harvey, “Golf ball aerodynamics”, Aeronaut., 27 (1976) 112-122.
Jin Choi, Woo-Pyung Jeon, and Haecheon Choi, “Mechanism of drag reduction by dimples on a sphere”, Physics of Fluids, 18 (2006) 041702.
Rabindra D. Mehta, “Aerodynamics of sports balls”, Ann. Rev. Fluid Mech., 17 (1983) 151-189.
Jeffrey Ryan Kensrud, “Determining aerodynamic properties of sports ball in situ”, Master of Science in Mechanical Engineering Thesis, Washington State University, 2010.
Koechner, W., Solid-state Laser Engineering (6^{th} Edition), Springer, Berlin (2005).
Kastrati, Adnan, Julinda Mehilli, Josef Dirschinger, Franz Dotzer, Helmut Schuhlen, Franz-Josef Neumann, Martin Fleckenstein, Conrad Pfafferott, Melchior Seyfarth, and Albert Schomig. “Intracoronary stenting and angiographic results.” Circulation 103, no. 23 (2001): 2816-2821.
Willstrand, O., Intensity distribution conversion from Gaussian to Top-Hat in a single-mode fiber connector (Master's Thesis) inUniversity of 18 (2006), 041702. Lund Reports on Atomic Physics, (2013).
Paschotta, R. (2010). Fluence. www.rp-photonics.com/fluence.html.
Tadashi Kokubo, Hiroaki Takadama., “How useful is SBF in predicting in vivo bone bioactivity?”, Biomaterials, 27, (2006): 2907-2915.
Silva, Carlos, and Doseo Park. “Optimization of fin performance in a laminar channel flow through dimpled surfaces.” Journal of Heat Transfer 131, no. 2 (2009): 021702.
Soltani Bozchalooi, A. Careaga Houck, J. M. AlGhamdi, and K. Youcef-Toumi, “Design and control of multi-actuated atomic force microscope for large-range and high-speed imaging”, Ultramicroscopy, 160 (2016): 213-224.
Sangtarash, Farhad, and Hossein Shokuhmand. “Experimental and numerical investigation of heat transfer, temperature distribution, and louver efficiency on the dimpled louvers fin banks.” Advances in Mechanical Engineering 7, (2015): 1687814015573825

What is claimed is:

1. A method for laser-assisted preparation of a dimpled stent, comprising the steps of:
(a) directing a laser beam at one or more targeted surface areas of a stent material using an assigned laser track to produce one or more dimples at the targeted surface areas, wherein the laser track is assigned a heat flux boundary with a moving laser beam defined by the equation:
$$k [ ( \frac{\partial T}{\partial x} ) + ( \frac{\partial T}{\partial y} ) + ( \frac{\partial T}{\partial z} ) ] = {P}_{X} - \{ h [ T - {T}_{0} ] + ɛ σ [ {T}^{4} - {T}_{0}^{4} ] \}$$
wherein k is thermal conductivity, h is heat transfer coefficient, ε is emissivity, σ is Stefan-Boltzmann constant, T is temperature, T₀ is ambient temperature, x is an X-coordinate in a three dimensional space, y is a Y-coordinate in a three dimensional space, z is a Z-coordinate in a three dimensional space, and Pₓ is a three-dimensional Gaussian laser power intensity distribution; and
(b) forming the stent material into a cylindrical shape around a central axis, wherein the one or more dimples protrude outwardly away from the central axis, to produce a dimpled stent.

2. The method of claim 1, wherein Pₓ is P_{g}, wherein P_{g} is Gaussian heat flux, and wherein P_{g} is defined by the equation:
$${P}_{g} = \frac{{P}_{0}}{π {r}_{0}^{2}} {exp}^{- ⌊ \frac{{x}^{2} + {y}^{2}}{{r}_{0}^{2}} ⌋}$$
wherein P₀ is laser input power, r₀ is radius of the laser beam at which laser power transverse intensity decreases to 1/e², and wherein x, y, z are Cartesian coordinates with y along a center axis of the beam and with x and z in a plane orthogonal to the center axis of the beam and wherein beam intensity distribution is axisymmetric in a x-z plane.

3. The method of claim 1, wherein Pₓ is Pₜₕ, wherein Pₜₕ is top hat heat flux, and wherein Pₜₕ is defined by the equation:
$${P}_{th} = \frac{{P}_{0}}{π {r}_{0}^{2}} {exp}^{- \frac{{( {x}^{2} + {y}^{2} )}^{n}}{{r}_{0}^{2}}}$$
wherein n→∞, P₀ is laser input power, r₀ is radius of the laser beam at which laser power transverse intensity decreases to 1/e², and wherein x, y, z are Cartesian coordinates with y along a center axis of the beam and with x and z in a plane orthogonal to the center axis of the beam and wherein beam intensity distribution is axisymmetric in a x-z plane.

4. The method of claim 1, wherein Pₓ is P_{db}, wherein P_{db} is dumbbell heat flux, and wherein P_{db} is defined by the equation:
$${P}_{db} = \frac{2 {P}_{0}}{π {r}_{0}^{2}} {( \frac{x + y}{{r}_{0}} )}^{2} {exp}^{- \frac{{( {x}^{2} + {y}^{2} )}^{n}}{{r}_{0}^{2}}}$$
wherein P₀ is laser input power, r₀ is radius of the laser beam at which laser power transverse intensity decreases to 1/e², and wherein x, y, z are Cartesian coordinates with y along a center axis of the beam and with x and z in a plane orthogonal to the center axis of the beam and wherein beam intensity distribution is axisymmetric in a x-z plane.

5. The method of claim 1, wherein the stent material is a titanium alloy, cobalt-chrome alloy, or stainless steel.

6. The method of claim 1, further comprising choosing a laser to generate a laser beam, wherein the laser is a Neodymium-Doped Yttrium Aluminum Garnet (Nd:Y₃Al₅O₁₂) laser.

7. The method of claim 1, further comprising choosing a laser to generate a laser beam, wherein the laser operates at a laser power of 500 W to 1800 W.

8. The method of claim 1, further comprising the step of using a Multiphysics computational model to predict and define features of the one or more dimples produced at the targeted surface areas, wherein a selective governing equation based on heat transfer for the Multiphysics computational model is defined by the equation:
$$ρ {{C}_{p} [ \frac{\partial T}{\partial L} ]}_{( x , y , z )} = k \{ {[ \frac{{\partial }^{2} T}{\partial {x}^{2}} ]}_{( y , z , t )} + {[ \frac{{\partial }^{2} T}{\partial {y}^{2}} ]}_{( z , x , t )} + {[ \frac{{\partial }^{2} T}{\partial {z}^{2}} ]}_{( x , y , t )} \}$$
wherein k is the thermal conductivity, Cₚ is specific heat and ρ is density of the stent material.

9. The method of claim 8, wherein width and depth of the one or more dimples produced at the targeted surface areas are determined based on the Multiphysics computational model.

10. The dimpled stent produced by the method of claim 1.

11. A dimpled stent, comprising:
circumferential walls enclosing a cylindrical inner space surrounding a central axis, wherein the circumferential walls comprise one or more laser produced dimples protruding outwardly away from the central axis to create one or more dimpled spaces in outer edges of the cylindrical inner space.

12. The dimpled stent of claim 11, wherein the dimpled stent is about 30 mm long and about 3.5 mm in diameter, wherein the circumferential walls are about 0.1 mm thick, and wherein the dimples have a depth of about 0.12 mm and a width of about 0.6 mm.

13. A dimpled stent, comprising:
a portion of stent material comprising adjacent rows of dimples protruding outwardly from the stent material, wherein each row of dimples comprises consecutive dimples, wherein each dimple has a depth d and a width w, wherein the dimples have a pitch P and a pitch R, wherein pitch P is spacing between centers of two consecutive dimples in a given row of dimples and P ranges from 1.8 w to 2.2 w, and wherein pitch R is spacing between centers of two dimples in two adjacent rows of dimples and R ranges from 1.8 w to 2.2 w.

14. The dimpled stent of claim 13, wherein each dimple has a depth between about 0.25 mm and 0.57 mm and a width between about 0.9 mm and 3.2 mm.

15. The dimpled stent of claim 13, wherein the portion of stent material is formed into a cylindrical tube shape wherein the dimples protrude outwardly from the cylindrical tube shape.

16. A dimpled stent, comprising:
a portion of stent material comprising adjacent rows of dimples protruding outwardly from the stent material, wherein each row of dimples comprises consecutive dimples, wherein each dimple has a depth d and a width w or w′, wherein the dimples have a pitch P and a pitch R, wherein pitch P is spacing between centers of two consecutive dimples in a given row of dimples and P ranges from 1.8 times (w+w′) and 2.2 times (w+w′), and wherein pitch R is spacing between centers of two dimples in two adjacent rows of dimples and R ranges from 1.8 w and 2.2 w.

17. The dimpled stent of claim 16, wherein each dimple has a depth between about 0.25 mm and 0.57 mm and a width between about 0.9 mm and 3.2 mm.

18. The dimpled stent of claim 16, wherein the portion of stent material is formed into a cylindrical tube shape wherein the dimples protrude outwardly from the cylindrical tube shape.
